(19) United States
(12) Patent Application Publication
(10) Pub. No.: US 2020/0350076 A1
(43) Pub. Date: Nov. 5, 2020

(54) Systems and Methods for Clinical Curation of Crowdsourced Data

(71) Applicant: Pear Therapeutics, Inc., Boston, MA (US)

(72) Inventors: Michael Brown, Arlington, MA (US); Kenneth R. Weingardt, South San Francisco, CA (US); Jillian Christine Ahrens, Kapolei, HI (US); Brent Paul Kersanske, Stoneham, MA (US)

(73) Assignee: Pear Therapeutics, Inc., Boston, MA (US)

(21) Appl. No.: 16/863,228

(22) Filed: Apr. 30, 2020

Related U.S. Application Data
(60) Provisional application No. 62/840,656, filed on Apr. 30, 2019.

(51) Int. Cl. G16H 50/30; G16H 50/70; G16H 50/20; G16H 10/60; G16H 70/00; G08B 21/18; G08B 21/02

(52) U.S. Cl. CPC G16H 50/30; G16H 50/70; G16H 50/20; G08B 21/02; G16H 70/00; G08B 21/182; G16H 10/60

(57) ABSTRACT

A method comprises obtaining input data from a patient device associated with a patient, the input data including free text data generated by the patient. The input data is analyzed to determine whether the input data meets predetermined relevancy criteria. The input data is compared to clinical data in a clinically curated database to generate comparison data. Based on the comparison data, the method comprises performing at least one of the following curation operations: (i) adding the input data to the clinically curated database when the input data meets predetermined relevancy criteria and when the comparison data indicates that the input data is sufficiently different from the clinical data; (ii) merging the input data with the clinical data when the input data meets predetermined relevancy criteria and when the comparison data indicates that the input data is sufficiently similar to the clinical data; and (iii) taking no action when it is determined that the input data does not meet predetermined relevancy criteria.

### CROSS REFERENCE TO RELATED APPLICATIONS

This U.S. patent application claims priority under 35 U.S.C. § 119(e) to U.S. Provisional Application 62/840,656, filed on Apr. 30, 2019. The disclosure of this prior application is considered part of the disclosure of this application and is hereby incorporated by reference in its entirety.

### FIELD

The present disclosure relates, generally, to the treatment of serious medical conditions and, more particularly, to systems and methods for implementing and managing a clinically curated database for the treatment of serious medical conditions.

### BACKGROUND

The information provided in this section is for the purpose of generally presenting the context of the disclosure. Work of the presently named inventors, to the extent it is described in this section, as well as aspects of the description that may not otherwise qualify as prior art at the time of filing, are neither expressly nor impliedly admitted as prior art against the present disclosure.

Drug therapy has played a significant role in the treatment of various medical diseases and disorders. Traditional drug therapy involves the administration of pharmaceuticals and the like. Examples of conventional pharmaceuticals may include small-molecule drugs, which are usually derived from chemical synthesis, and biopharmaceuticals, which may include recombinant proteins, vaccines, blood products used therapeutically gene therapy, monoclonal antibodies, cell therapy, and the like.

While drug therapy has proven to be an effective mechanism for treating certain diseases and disorders, it is not without drawbacks. For example, pharmaceuticals are known to come with certain, frequently undesirable, side-effects. In addition, pharmaceuticals are often costly—sometimes prohibitively so.

Accordingly, digital solutions for treating various medical diseases and disorders have emerged as a compliment, or alternative, to conventional drug therapy techniques. Such digital solutions (e.g., digital therapeutics, mobile health applications, etc.) may solicit information from users (e.g., patients in the case of a prescription digital therapeutic or “PDT”) thereof. Such information may include, by way of example and not limitation, information concerning the user's mental state (e.g., feelings the user is or has experienced) and/or physical state (e.g., physical symptoms associated with mental or physical health conditions).

Conventional digital solutions frequently presented the user with a fixed set of selectable responses to a given query. For example, in association with a query such as “how are you feeling,” conventional digital solutions may present the user with a set of selectable options such as “happy,” “sad,” “scared,” “tired,” “in pain,” “sleepy,” etc. However, these pre-selected, fixed responses often fail to adequately capture the user's mental or physical state.

Accordingly, systems and methods for implementing and managing a clinically curated database for the treatment of serious medical conditions may be needed.

### BRIEF DESCRIPTION OF THE FIGURES

Reference will now be made to the accompanying Figures, which are not necessarily drawn to scale, and wherein:

FIG. 1 is a schematic view of a system for implementing and managing a clinically curated database in accordance with an exemplary embodiment of the present disclosure;

FIG. 2 is a functional block diagram of a system for implementing and managing a clinically curated database in accordance with an exemplary embodiment of the present disclosure;

FIG. 3 is a patient device displaying a first graphical user interface executed by at least a portion of the system of FIG. 2;

FIG. 4 is a patient device displaying a second graphical user interface executed by at least a portion of the system of FIG. 2;

FIGS. 5A-5D are a process for curating a clinically curated database using the system of FIG. 2;

FIG. 6 is a flowchart illustrating a method performed by the system of FIG. 2; and

FIG. 7 is a schematic view of an electronic device for implementing and managing a clinically curated database in accordance with an exemplary embodiment of the present disclosure.

Like reference symbols in the various drawings indicate like elements.

### SUMMARY

One aspect of the disclosure provides a system comprising data processing hardware and memory hardware in communication with the data processing hardware. The memory hardware stores instructions that when executed on the data processing hardware cause the data processing hardware to perform operations comprising obtaining input data from a patient device associated with a patient, the input data including free text data generated by the patient. The operations comprise analyzing the input data to determine whether the input data meets predetermined relevancy criteria. The operations comprise comparing the input data to clinical data in a clinically curated database to generate comparison data, and based on the comparison data, performing at least one of the following curation operations: (i) adding the input data to the clinically curated database when the input data meets predetermined relevancy criteria and when the comparison data indicates that the input data is sufficiently different from the clinical data; (ii) merging the input data with the clinical data when the input data meets predetermined relevancy criteria and when the comparison data indicates that the input data is sufficiently similar to the clinical data; and (iii) taking no action when it is determined that the input data does not meet predetermined relevancy criteria.

Implementations of the disclosure may include one or more of the following optional features. In some implementations, the operations further comprise analyzing the input data to determine a likelihood of an adverse event, and assigning a risk-assessment value to the input data corresponding to the likelihood that the input data indicates an adverse event has occurred or is going to occur. When the risk-assessment value associated with the input data exceeds a predetermined threshold, the operations may further comprise executing a mechanism of action to address the adverse event.

The mechanism of action may include sending an alert to a healthcare provider device associated with a healthcare provider supervising the patient, the alert indicating that an adverse event has occurred or is going to occur. The mechanism of action may include sending an alert to a call center device associated with a call center, the alert indicating that an adverse event has occurred or is going to occur and the alert providing instructions to the call center to contact the patient via the patient device. The mechanism of action may include sending an alert to the patient device, the alert providing information to the patient to address the adverse event.

Analyzing the input data and comparing the input data to the clinical data may be performed by implementing artificial intelligence. The artificial intelligence may be supervised by a healthcare professional. The artificial intelligence may include unsupervised machine learning.

The input data may be input in response to an inquiry, and the predetermined relevancy criteria may be satisfied when the input data is responsive to the inquiry.

Another aspect of the disclosure provides a method comprising obtaining, via one or more processors, input data from a patient device associated with a patient, the input data including free text data generated by the patient. The input data is analyzed, via the one or more processors, to determine whether the input data meets predetermined relevancy criteria. The input data is compared, via the one or more processors, to clinical data in a clinically curated database to generate comparison data. Based on the comparison data, the method comprises performing at least one of the following curation operations: (i) adding the input data to the clinically curated database when the input data meets predetermined relevancy criteria and when the comparison data indicates that the input data is sufficiently different from the clinical data; (ii) merging the input data with the clinical data when the input data meets predetermined relevancy criteria and when the comparison data indicates that the input data is sufficiently similar to the clinical data; and (iii) taking no action when it is determined that the input data does not meet predetermined relevancy criteria. This aspect may include one or more of the following optional features.

In some implementations, the method further comprises analyzing the input data to determine a likelihood of an adverse event, and assigning a risk-assessment value to the input data corresponding to the likelihood that the input data indicates an adverse event has occurred or is going to occur. The method may further comprise executing a mechanism of action to address the adverse event, when the risk-assessment value associated with the input data exceeds a predetermined threshold.

The mechanism of action may include sending an alert to a healthcare provider device associated with a healthcare provider supervising the patient, the alert indicating that an adverse event has occurred or is going to occur. The mechanism of action may include sending an alert to a call center device associated with a call center, the alert indicating that an adverse event has occurred or is going to occur and the alert providing instructions to the call center to contact the patient via the patient device. The mechanism of action may include sending an alert to the patient device, the alert providing information to the patient to address the adverse event.

Analyzing the input data and comparing the input data to the clinical data may be performed by implementing artificial intelligence. The artificial intelligence may be supervised by a healthcare professional. The artificial intelligence may include unsupervised machine learning.

The input data may be input in response to an inquiry, and the predetermined relevancy criteria may be satisfied when the input data is responsive to the inquiry.

The details of one or more implementations of the disclosure are set forth in the accompanying drawings and the description below. Other aspects, features, and advantages will be apparent from the description and drawings, and from the claims.

### DETAILED DESCRIPTION

Some implementations of the disclosed technology will be described more fully with reference to the accompanying drawings. This disclosed technology may, however, be embodied in many different forms and should not be construed as limited to the implementations set forth herein.

Example implementations of the disclosed technology provide systems and methods for implementing and managing a clinically curated database for the treatment of serious medical conditions.

For the treatment of certain medical conditions or indications, such as, for example, opioid abuse, multiple sclerosis, depression, etc., input from a patient may aid in tailoring the treatment to the needs of that specific patient by providing information to the medical professionals controlling the treatment. Such input may be in the form of responses to multiple-choice questions, responses to open-ended questions, unstructured free text, etc. Additionally, the patient input may prompt and demonstrate to the patient similar input of other patients from a clinically curated database, which may cause the patient to feel like they are not alone in experiencing their specific thoughts and emotions.

In some examples, patients suffering from certain medical conditions may experience mental health symptoms, which may be a natural reaction to the unpredictable course of certain medical conditions, e.g., a disabling chronic disease. Mental health symptoms may include depression, anxiety, mood swings, etc. Patients with certain medical conditions may be predisposed for mental health symptoms due to psychological risk factors such as inadequate coping or insufficient social support, as well as biological processes such as changes in brain structure.

There is no association between the severity of symptoms and likelihood of a patient experiencing mental health symptoms; any patient with a medical condition can experience mental health symptoms at any point, but a variety of factors may influence mental health symptoms in patients. A patient's initial diagnosis may be followed by a period of mental health symptoms. Patients may also experience mental health symptoms due to the physical symptoms associated with a certain medical condition. For example, a patient suffering from fatigue may be depleted of emotional energy required to fight mental health symptoms. Furthermore, a patient's high level of uncertainty about new symptoms and the future may cause patients to experience mental health symptoms. Physiological causes, such as damage to the central nervous system, and chemical changes, such as expression of pro-inflammatory protein molecules involved in cell-to-cell communications, may cause patients to experience mental health symptoms as well. Medication side effects can worsen mental health symptoms. Steroids, for example, can cause euphoria in the short term, followed by mental health symptoms once the euphoria has stopped.

Mental health symptoms significantly affect the mood of a patient suffering from certain medical conditions, thereby negatively affecting the patient's quality of life. Patients may prioritize physical health over emotional health and leave mental health symptoms untreated, which may lead to reduced quality of life and impaired cognitive function. For example, patients experiencing mental health symptoms may seek to withdraw from daily life activities, resulting in reduced social stimulation. Patients may also experience an increased risk of suicide.

Current treatment options for mental health symptoms in patients with certain medical conditions generally include medication and face-to-face therapy with a clinician. However, these treatment options may be supplemented with more effective patient input.

Example implementations of the disclosed technology will now be described with reference to the accompanying figures.

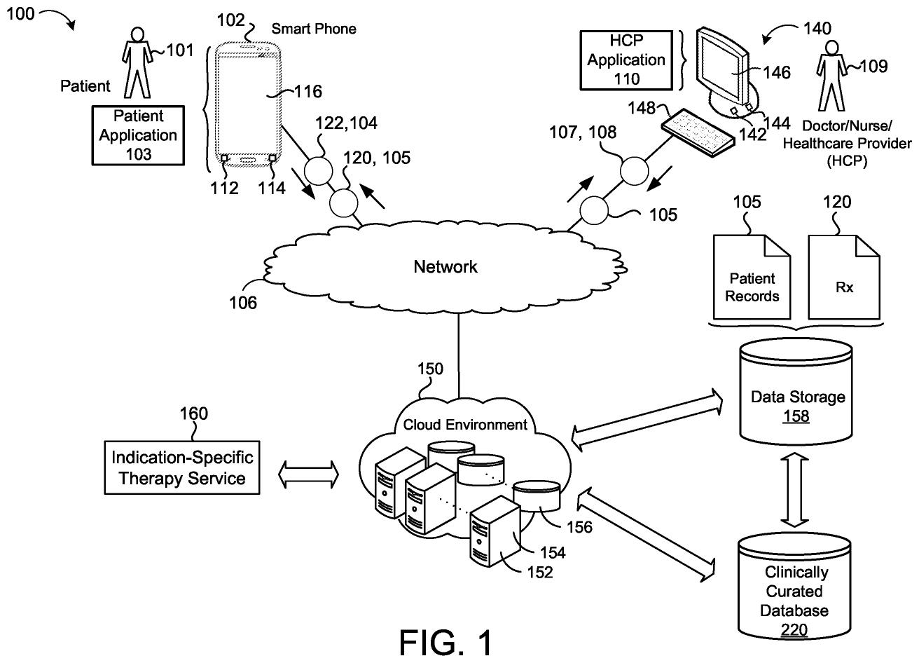

Referring to FIG. 1, in some implementations, a therapy prescription system 100 provides a patient 101 access to a prescription digital therapeutic 120 prescribed to the patient 101 and monitors events associated with the patient's 101 interaction with the prescription digital therapeutic 120. Although the digital therapeutic 120 is described herein as being a “prescription” digital therapeutic, it is understood that, according to some implementations, the digital therapeutic 120 will not require a prescription from a clinician. Rather, in such implementations, the digital therapeutic 120 may be available to a patient without a prescription, and the digital therapeutic 120 nonetheless otherwise functions in accordance with the description of the prescription digital therapeutic 120 described herein. According to implementations in which the digital therapeutic 120 is not prescribed, the person using or being administered the digital therapeutic may be referred to as a “user.” A “user” may include a patient 101 or any other person using or being administered the digital therapeutic 120, irrespective of whether the digital therapeutic 120 was prescribed to that person.

As used herein, a digital therapy may also be referred to as a digital-therapeutic configured to deliver evidence-based psychosocial intervention techniques for treating a patient with a particular disease or disorder, as well as symptoms and/or behaviors associated with the particular disease or disorder. As one example, the patient 101 may be diagnosed with a chronic disease and the prescription digital therapeutic 120 may be specifically tailored for addressing one or more depressive symptoms associated with the chronic disease that the patient 101 may experience. An authorized healthcare provider (HCP) 109 (e.g., a doctor, nurse, etc.) supervising the patient 101 may prescribe the patient 101 the prescription digital therapeutic 120 designed to help the patient 101 identify feelings the patient 101 is experiencing and modify dysfunction emotions, behaviors, and thoughts in order to treat depressive symptoms in the patient 101. The HCP 109 may include a physician, nurse, clinician, or other qualified health professionals.

In some examples, the system 100 includes a network 106, a patient device 102, an HCP system 140, and a medical indication-specific therapy service 160. For example, the therapy service 160 may be related to a specific indication such as opioid abuse, multiple sclerosis, depression, etc. The network 106 provides access to cloud computing resources 150 (e.g., distributed system) that execute the therapy service 160 to provide for the performance of services on remote devices. Accordingly, the network 106 allows for interaction between patients 101 and HCPs 109 with the therapy service 160. For instance, the therapy service 160 may provide the patient 101 access to the prescription digital therapeutic 120 and receive event data 122 inputted by the patient 101 associated with the patient's 101 interaction with the prescription digital therapeutic 120. In turn, the therapy service 160 may store the event data 122 on a storage resource 156.

The network 106 may include any type of network that allows sending and receiving communication signals, such as a wireless telecommunication network, a cellular telephone network, a time division multiple access (TDMA) network, a code division multiple access (CDMA) network, Global system for mobile communications (GSM), a third generation (3G) network, fourth generation (4G) network, a satellite communications network, and other communication networks. The network 106 may include one or more of a Wide Area Network (WAN), a Local Area Network (LAN), and a Personal Area Network (PAN). In some examples, the network 106 includes a combination of data networks, telecommunication networks, and a combination of data and telecommunication networks. The patient device 102, the HCP system 140, and the therapy service 160 communicate with each other by sending and receiving signals (wired or wireless) via the network 106. In some examples, the network 106 provides access to cloud computing resources, which may be elastic/on-demand computing and/or storage resources 156 available over the network 106. The term “cloud” services generally refers to a service performed not locally on a user's device, but rather delivered from one or more remote devices accessible via one or more networks 106.

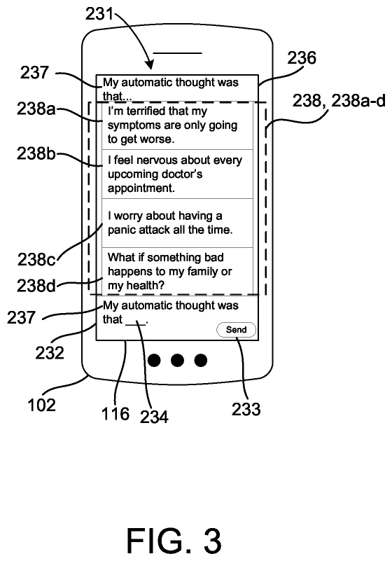

The patient device 102 may include, but is not limited to, a portable electronic device (e.g., smartphone, cellular phone, personal digital assistant, personal computer, wireless tablet device, or a wearable device), a desktop computer, or any other electronic device capable of sending and receiving information via the network 106. The patient device 102 includes data processing hardware 112 (a computing device that executes instructions), memory hardware 114, and a display 116 in communication with the data processing hardware 112. In some examples, the patient device 102 includes a keyboard, mouse, microphones, and/or a camera for allowing the patient 101 to input data. In addition to or in lieu of the display 116, the patient device 102 may include one or more speakers to output audio data to the patient 101. For instance, audible alerts may be output by the speaker to notify the patient 101 about some time sensitive event associated with the prescription digital therapeutic 120. In some implementations, the patient device 102 executes a patient application 103 (or accesses a web-based patient application) for establishing a connection with the therapy service 160 to access the prescription digital therapeutic 120. For instance, the patient 101 may have access to the patient application 103 for a duration (e.g., 3 months) of the prescription digital therapeutic 120 prescribed to the patient 101. Here, the patient device 102 may launch the patient application 103 by initially providing an access code 104 when the prescription digital therapeutic 120 is prescribed by the HCP 109 that allows the patient 101 to access content associated with the prescription digital therapeutic 120 from the therapy service 160 that is specifically tailored for treating/addressing one or more symptoms associated with the specific indication that the patient 101 may be experiencing. The patient application 103, when executing on the data processing hardware 112 of the patient device 102, is configured to display a variety of graphical user interfaces (GUIs) (e.g., a patient input GUI 231 as shown in FIG. 3) on the display 116 of the patient device 102 that, among other things, allow the patient 101 to input event data 122 associated with particular feelings the patient is experiencing, solicit information from the patient 101, and present journal entries for the patient 101 to view.

The storage resources 156 may provide data storage 158 for storing the event data 122 received from the patient 101 in a corresponding patient record 105 as well as the prescription digital therapeutic 120 prescribed to the patient 101. In some implementations, the data storage 158 is in communication with a clinically curated database 220 that is in communication with the cloud computing system 150. For example, the data storage 158 may share the patient records 105, the prescription digital therapeutic 120, and/or any other suitable information with the clinically curated database 220, and the clinically curated database 220 may share clinically curated entries and/or any other suitable information with the data storage 158. In other implementations, the data storage 158 stores the clinically curated database 220. The patient record 105 may be encrypted while stored on the data storage 158 so that any information identifying the patient 101 is anonymized, but may later be decrypted when the patient 101 or supervising HCP 109 requests the patient record 105 (assuming the requester is authorized/authenticated to access the patient record 105). All data transmitted over the network 106 between the patient device 102 and the cloud computing system 150 may be encrypted and sent over secure communication channels. For instance, the patient application 103 may encrypt the event data 122 before transmitting to the therapy service 160 via the HTTPS protocol and decrypt a patient record 105 received from the therapy service 160. When network connectivity is not available, the patient application 103 may store the event data 122 in an encrypted queue within the memory hardware 114 until network connectivity is available.

The HCP system 140 may be located at a clinic, doctor's office, or facility administered by the HCP 109 and includes data processing hardware 142, memory hardware 144, and a display 146. The memory hardware 144 and the display 146 are in communication with the data processing hardware 142. For instance, the data processing hardware 142 may reside on a desktop computer or portable electronic device for allowing the HCP 109 to input and retrieve data to and from the therapy service 160. In some examples, the HCP 109 may initially onboard some or all of patient data 107 at the time of prescribing the prescription digital therapeutic 120 to the patient 101. The HCP system 140 includes a keyboard 148, mouse, microphones, speakers and/or a camera. In some implementations, the HCP system 140 (i.e., via the data processing hardware 142) executes a HCP application 110 (or accesses a web-based patient application) for establishing a connection with the therapy service 160 to input and retrieve data therefrom. For instance, the HCP system 140 may be able to access the anonymized patient record 105 securely stored by the therapy service 160 on the storage resources 156 by providing an authentication token 108 validating that the HCP 109 is supervising the patient 101 and authorized to access the corresponding patient record 105. The authentication token 108 may identify the particular patient 101 associated with the patient record 105 that the HCP system 140 is permitted to obtain from the therapy service 160. The patient record 105 may include time-stamped event data 122 indicating the patient's interaction with the prescription digital therapeutic 120 through the patient application 103 executing on the patient device 102.

The cloud computing resources 150 may be a distributed system (e.g., remote environment) having scalable/elastic resources 152. The resources 152 include computing resources 154 (e.g., data processing hardware) and/or the storage resources 156 (e.g., memory hardware). The cloud computing resources 150 execute the therapy service 160 for facilitating communications with the patient device 102 and the HCP system 140, and storing data on the storage resources 156 within the data storage 158 and storing data on the clinically curated database 220. In some examples, the therapy service 160, the data storage 158, and the clinically curated database 220 reside on a standalone computing device. The therapy service 160 may provide the patient 101 with the patient application 103 (e.g., a mobile application, a web-site application, or a downloadable program that includes a set of instructions) executable on the data processing hardware 112 and accessible through the network 106 via the patient device 102 when the patient 101 provides a valid access code 104. Similarly, the therapy service 160 may provide the HCP 109 with the HCP application 110 (e.g., a mobile application, a web-site application, or a downloadable program that includes a set of instructions) executable on the data processing hardware 142 and accessible through the network 106 via the HCP system 140.

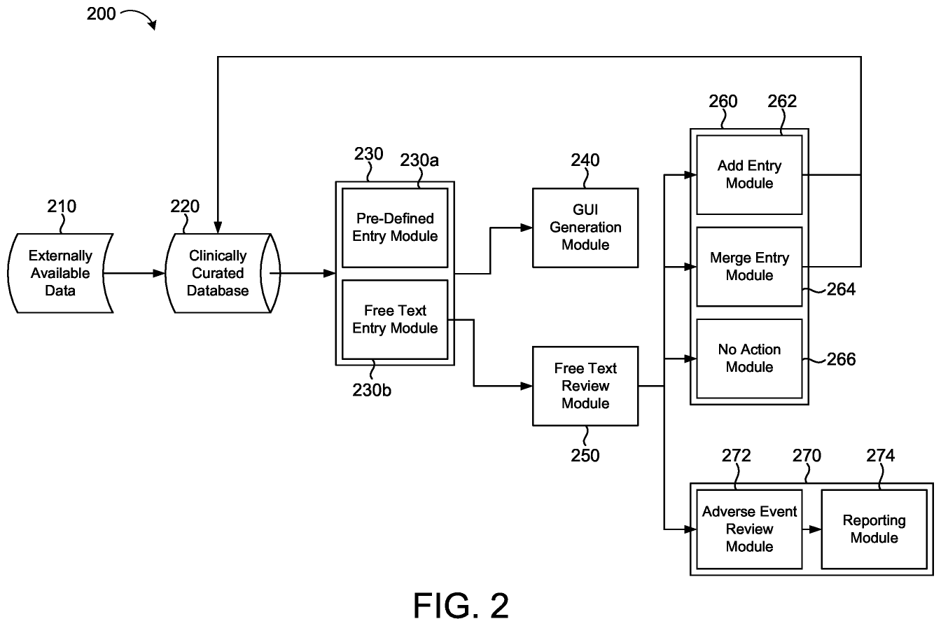

Referring to FIG. 2, a diagram illustrating a system 200 for implementing and managing a clinically curated database 220 is shown in accordance with an exemplary implementation of the present disclosure. According to one example, aspects of the system 200 may be executed by the computing resources 154 of the cloud computing system 150. In another example, aspects of the system 200 may be executed by an electronic device, such as the data processing hardware 112 of the patient device 102. In yet another example, aspects of the system 200 may be executed by some combination of the computing resources 154 and the data processing hardware 112. In some implementations, externally available data 210 is obtained (e.g., fetched or received) by the clinically curated database 220. The externally available data 210 may be obtained from a variety of sources, such as, for example, the Federal Drug Administration (FDA), the World Health Organization (WHO), the International Classification of Diseases: Tenth Revision (ICD-10), etc. As set forth above, the clinically curated database 220 may be in communication with the cloud computing resources 150 or stored on the data storage 158 of the cloud computing resources 150. In other implementations, the clinically curated database 220 may be stored on the memory hardware 114 of the patient device 102, the memory hardware 144 of the HCP system 140, or any other suitable storage location.

The system 200 includes an input module 230 having a pre-defined entry module 230a and a free text entry module 230b. The input module 230 may be executed by the patient device 102, i.e., the data processing hardware 112 in conjunction with the display 116 and/or other peripherals, such as the microphone, speakers, mouse, keyboard, camera, etc., of the patient device 102. The input module 230 is in communication with the clinically curated database 220 to obtain (e.g., fetch or receive) data from the clinically curated database 220.

Referring to FIGS. 2 and 3, in some examples, the display 116 of the patient device 102 includes a touch screen displaying a patient input GUI 231. The data processing hardware 112 may execute GUI software adapted to facilitate human interaction with the patient input GUI 231. Described in greater detail below, the patient 101 may provide user-selections indicating selection to interact with the patient input GUI 231. As used herein, a user-selection may be directed to a UI control that includes any displayed element or component of the patient input GUI 231 displayed on the display 116. As such, user-selection indicating selection of a UI control may permit the patient 101 to provide input, view data, and/or otherwise interact with the patient input GUI 231. Example UI controls include buttons, drop down menus, menu items, tap-and-hold functionality, etc.

The patient input GUI 231 displays a free text data entry element 232, a data entry header element 236, and a plurality of pre-defined entries 238 including individual exemplary entries 238a-d. In some examples, the data entry header element 236 and the free text data entry element 232 may each include an entry prompt 237. The entry prompt 237 may be a question or statement intended to elicit a response from the patient 101. For example, the entry prompt 237 may read “My automatic thought was that . . . ,” prompting the patient 101 to respond with their automatic thought. Each entry prompt 237 may be associated with the plurality of pre-defined entries 238 that are selectable responses to the entry prompt 237. For example, the pre-defined entry module 230a may determine when and which of the pre-defined entries 238 are selected by the patient 101. The entry prompt 237 and its associated pre-defined entries 238 are retrieved from the clinically curated database 220 and displayed in the patient input GUI 231. For example, as shown in FIG. 3, one of the pre-defined entries 238c may read “I worry about having a panic attack all the time” in response to the entry prompt 237, “My automatic thought was that . . . .” As will become apparent, the pre-defined entries 238 may be based, at least in part, on the externally available data 210, free text responses from other patients that have been reviewed and added to the clinically curated database 220, or combinations thereof.

In some implementations, the patient input GUI 231 may display a string of free text data 234 reflecting a patient's typed or spoken response to the entry prompt 237. As shown in FIG. 3, the string of free text data 234 may be entered in the underlined space following the entry prompt 237. The free text data entry element 232 allows the patient 101 to enter the string of free text data 234 by typing via a keyboard (not shown) in the free text data entry element 232 or speaking into a microphone of the patient device 102. The free text data 234 is displayed in the free text data entry element 232, and, in some embodiments, appended to the entry prompt 237.

The patient 101 may respond to the entry prompt 237 in several ways. According to one embodiment, the patient 101 may respond by selecting (e.g., through a touch gesture or other suitable input mechanism) one of the pre-defined entries 238 displayed on the display 116 of the patient device 102, as determined by the pre-defined entry module 230a. According to another embodiment, the patient 101 may respond by entering free text data 234 into the free text data entry element 232, for example, by typing into a keyboard (e.g., via a keyboard GUI that may pull up over at least a portion of the patient input GUI 231) or speaking into a microphone of the patient device 102, as determined by the free text entry module 230b. According to some examples, the patient may finalize or confirm their response to the entry prompt 237 by selecting a send button 233, as determined by the input module 230.

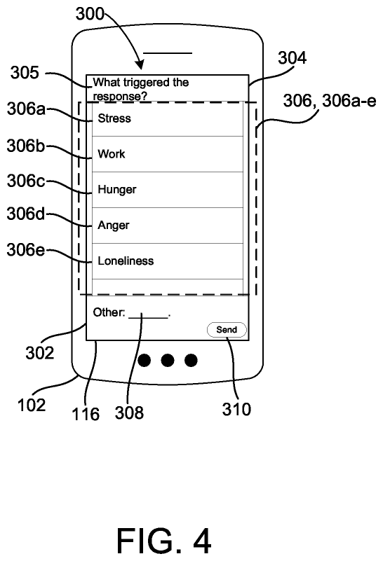

Referring to FIGS. 2 and 3, the input module 230 is in communication with a GUI generation module 240 that is configured to generate a GUI, such as the patient input GUI 231 (FIG. 3) or a patient trigger GUI 300 (FIG. 4), that is displayed on the display 116 of the patient device 102. Upon a user-selection indication indicating selection of one of the pre-defined entries 238, the GUI generation module 240 displays the selected one of the pre-defined entries 238 on the patient input GUI 231. For example, while not shown, the selected one of the pre-defined entries 238 may be highlighted, isolated, or identified in any suitable manner to indicate selection of that pre-defined entry 238. Upon a user-selection indication indicating selection of the entry prompt 237 and subsequent detection of entry of free text data 234, the GUI generation module 240 displays the free text data 234 and/or the entry prompt 237 on the patient input GUI 231. For example, while not shown, the free text data 234 may be highlighted, isolated, or identified in any suitable manner to indicate entry of the free text data 234.

The free text review module 250 is configured to review and analyze the free text data 234 to determine what further action should be taken in accordance with the free text data 234. For example, the free text review module 250 is configured to determine whether the free text data 234 meets predetermined relevancy criteria (i.e., whether the free text data 234 is responsive to the entry prompt 237) and whether the free text data 234 indicates a likelihood that an adverse event has occurred or is going to occur. According to some examples, the free text review module 250 is configured to implement artificial intelligence and/or machine learning (supervised or unsupervised) to determine what further action should be taken with regard to the free text data 234. Based on its determination, the free text review module 250 is configured to selectively pass the free text data 234 onto a clinical data curation module 260 for further processing.

The clinical data curation module 260 includes an add entry module 262, a merge entry module 264, and a no action module 266. The add entry module 262 is configured to add the free text data 234 to the clinically curated database 220 (e.g., as a new entry in the database 220) based on a determination that the free text data 234 satisfies predetermined criteria. Such predetermined criteria may include, but is not limited to: (i) relevancy of the free text data 234 (e.g., according to a calculated relevancy score), (ii) a determination that the free text data 234 contains sensitive or inappropriate content (e.g., based on a determination that the free text data 234 includes certain known words, such as swear words, profanity, or the like), (iii) a determination that the clinically curated database 220 already contains a similar entry (e.g., based on a comparison of the free text data 234 to pre-defined entries 238 in the clinically curated database 220 to generate comparison data), (iv) etc. For example, if the free text data 234 reads “I feel like I am a burden on my family,” and there are no similarly related pre-defined entries 238 in the clinically curated database 220, then the comparison data may indicate that the free text data 234 should be added to the clinically curated database 220, and the add entry module 262 may add the free text data 234 to the clinically curated database 220.

According to one example, the free text data 234 may be vectorized and compared to corresponding vector data associated with the pre-defined entries 238 in the clinically curated database 220 to generate the comparison data. According to this example, the add entry module 262 may determine that a given free text data 234 entry is sufficiently different from a given pre-defined entry 238 in the clinically curated database 220 if the vectors for the respective entries are outside of a predefined threshold. Such a determination may be made by artificial intelligence and/or machine learning (supervised or unsupervised). If it is determined that the free text data 234 is sufficiently different from one of the pre-defined entries 238 in the clinically curated database 220, the free text data 234 may be added to the clinically curated database 220 as a new entry by the add entry module 262.

The merge entry module 264 is configured to determine whether the free text data 234 is closely related to any pre-defined entries 238 in the clinically curated database 220. According to one example, the free text data 234 may be vectorized and compared to corresponding vector data associated with the pre-defined entries 238 in the clinically curated database 220 to generate the comparison data. According to this example, the merge entry module 264 may determine that a given free text data 234 entry is related to a given pre-defined entry (e.g., entry 238a) in the clinically curated database 220 if the vectors for the respective entries are within a predefined threshold. Such a determination may be made by artificial intelligence and/or machine learning (supervised or unsupervised). If it is determined that the free text data 234 is sufficiently related to one of the pre-defined entries 238 in the clinically curated database 220, the free text data 234 may be merged with the associated pre-defined entry (e.g., entry 238a) by the merge entry module 264. Similarity between the free text data 234 and the pre-defined entries 238 may be based, according to some examples, on the meaning of the free text data 234 and the meaning of the pre-defined entries 238. For example, if the free text data 234 reads “I'm scared” and one of the pre-defined entries 238 reads “I'm afraid,” the merge entry module 264 may merge the free text data 234 for “I'm scared” with the pre-defined entry corresponding to “I'm afraid.” As another example, the comparison data may indicate that the free text data 234 contains a typo or misspelling (e.g., “I'm afriad”), but is sufficiently similar to one of the pre-defined entries 238 (e.g., a pre-defined entry corresponding to “I'm afraid”), such that the merge entry module 264 merges this free text data 234 with the pre-defined entry corresponding to “I'm afraid.”

If it is determined that the free text data 234 does not meet the criteria to be added to the clinically curated database 220 or merged with one of the pre-defined entries 238 in the clinically curated database 220 (i.e., the free text data 234 does not meet predetermined relevancy criteria), then no action is taken at the no action module 266 and the free text data 234 stays as entered. For example, if the free text data 234 includes nonsensical text (e.g., “abcd1234,” “!@kfycn,” lplahsnxc,” etc.) or irrelevant text (i.e., text that is nonresponsive to the entry prompt 237, such as “The sky is blue”), the no action module 266 is configured to prevent the free text data 234 from being added to the clinically curated database 220.

Among other advantages, adding new data entries into the clinically curated database 220, merging data entries into the clinically curated database 220, and preventing entries from being added to the clinically curated database 220 may result in the clinically curated database 220 being populated with the most relevant results, resulting in improved outcomes for the patient 101.

According to some examples, upon analysis of the free text data 234, the free text review module 250 may determine that the free text data 234 implicates a possible adverse event for the patient 101 or others based on the content of the free text data 234. Such a determination may be made by artificial intelligence and/or machine learning (supervised or unsupervised). For example, the free text review module 250 may detect an adverse event condition based on the presence of certain keywords representative of possible harm to the patient 101 or others. An adverse event condition may be detected by the free text review module 250 if the free text data 234 reflects statements of harm such as “I want to kill myself,” “I want to harm myself,” “I want to harm others,” etc.

Upon a detection of an adverse event condition, the free text review module 250 may pass the free text data 234 on to a regulatory review module 270 for further processing. The regulatory review module 270 may include an adverse event review module 272 and a reporting module 274. The adverse event review module 272 is configured to determine the likelihood of an adverse event based on the free text data 234. This determination may be based on, for example, the presence of certain keywords in the free text data 234 (e.g., “kill,” “harm,” “hurt,” etc.), the presence of specific drug street names (e.g., “heroin,” “cocaine,” etc.), drug trade names (e.g., “Suboxone®”), and/or drug manufacturer names (e.g., “Big Pharma Corp.”) in the free text data 234, and/or a comparison of the free text data 234 to previously reviewed responses indicative of an adverse event. In some implementations, the determination of the likelihood of an adverse event may be based on established clinical measures to assess self-harm. For example, the adverse event review module 272 may analyze the free text data 234 and generate a risk-assessment value based on the free text data 234. In some implementations, generating a risk-assessment value may include comparing the free text data 234 against entries in a pre-existing database, such as an established clinical database. Such analysis and/or comparison may be conducted by artificial intelligence and/or machine learning (supervised or unsupervised). Upon a determination by the adverse event review module 272 that the free text data 234 is indicative of an adverse event, the adverse event review module 272 may trigger action by the reporting module 274.

The reporting module 274 is configured to execute a mechanism of action, such as, for example, sending an alert to the HCP system 140, sending an alert to the therapy service 160, or sending an alert and the phone number of the patient 101 to a suicide hotline, or other suitable call center, crisis hotline, etc., instructing the suicide hotline to contact the patient 101. Additionally or alternatively, the reporting procedures may follow processes set forth by organizations such as the FDA, the WHO, etc.

As described above, the patient input GUI 231 relates to an automatic thought of the patient 101. However, it should be understood that the patient input GUI 231 illustrates one exemplary GUI that may be displayed on the display 116, and other GUIs may likewise be displayed on the display 116 in a similar manner.

For example, referring to FIG. 4, a patient trigger GUI 300 may be executed by the input module 230 and displayed on the display 116 of the patient device 102. The patient trigger GUI 300 may display, via the input module 230, a free text data entry element 302, a data entry header element 304, and a plurality of pre-defined entries 306 including individual exemplary entries 306a-e. In some examples, the data entry header element 304 may include an entry prompt 305. The entry prompt 305 may be a question or statement intended to elicit a response from the patient 101, similar to the entry prompt 237. For example, the entry prompt 305 may read “What triggered the response?”, prompting the patient 101 to respond with a particular trigger. In some implementations, the response may be associated with a relapse (e.g., a drug or alcohol relapse), and the triggers may be associated with an event, activity, emotion, etc., that triggered the relapse.

Each entry prompt 305 may be associated with the plurality of pre-defined entries 306 that are selectable responses to the entry prompt 305. For example, the pre-defined entry module 230a may determine when and which of the pre-defined entries 306 are selected by the patient 101. The entry prompt 305 and its associated pre-defined entries 306 are retrieved from the clinically curated database 220 and displayed in the patient trigger GUI 300. For example, as shown in FIG. 4, a first pre-defined entry 306a may read “Stress,” a second pre-defined entry 306b may read “Work,” a third pre-defined entry 306c may read “Hunger,” a fourth pre-defined entry 306d may read “Anger,” and a fifth pre-defined entry 306e may read “Loneliness.” In addition to these triggers, any other suitable trigger is contemplated, such as tiredness, fatigue, social pressure, pain, boredom, etc. Similar to the pre-defined entries 238 above, the pre-defined entries 306 may be based, at least in part, on the externally available data 210, free text responses from other patients that have been reviewed and added to the clinically curated database 220, or combinations thereof.

In some implementations, the patient trigger GUI 300 may display, via the free text entry module 230b, a string of free text data 308 reflecting a patient's typed or spoken response to the entry prompt 305. As shown in FIG. 4, the string of free text data 308 may be entered in the underlined space following “Other.” The free text data entry element 302 allows the patient 101 to enter the string of free text data 308 by typing via a keyboard (not shown) in the free text data entry element 302 or speaking into a microphone of the patient device 102. The free text data 308 is displayed in the free text data entry element 302, and, in some embodiments, appended to the entry prompt 305. As set forth above with respect to the entry prompt 237, the patient 101 may respond to the entry prompt 305 in several ways, such as touch gestures, voice, etc. According to some examples, the patient may finalize or confirm their response to the entry prompt 305 by selecting a send button 310, as determined by the input module 230.

Similar to the description above with respect to the system 200 and the patient input GUI 231, the system 200 may likewise execute the GUI generation module 240, free text review module 250, clinical data curation module 260, and regulatory review module 270 with respect to information obtained from interaction between the patient 101 and the patient trigger GUI 300.

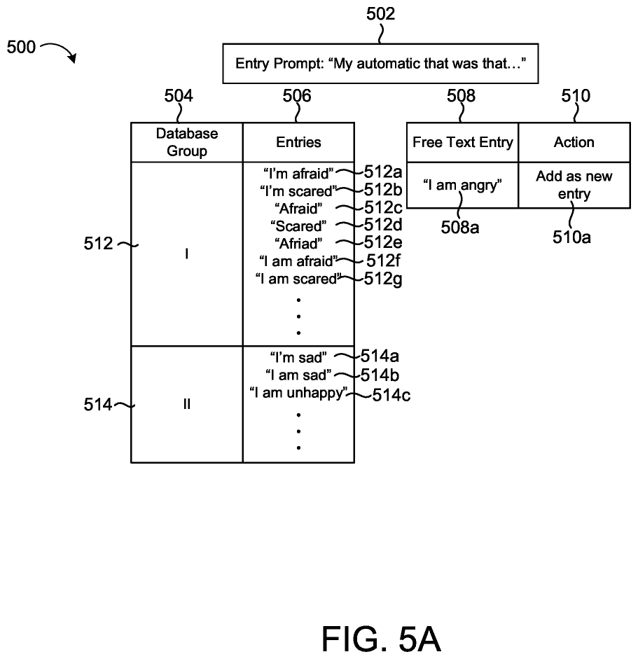

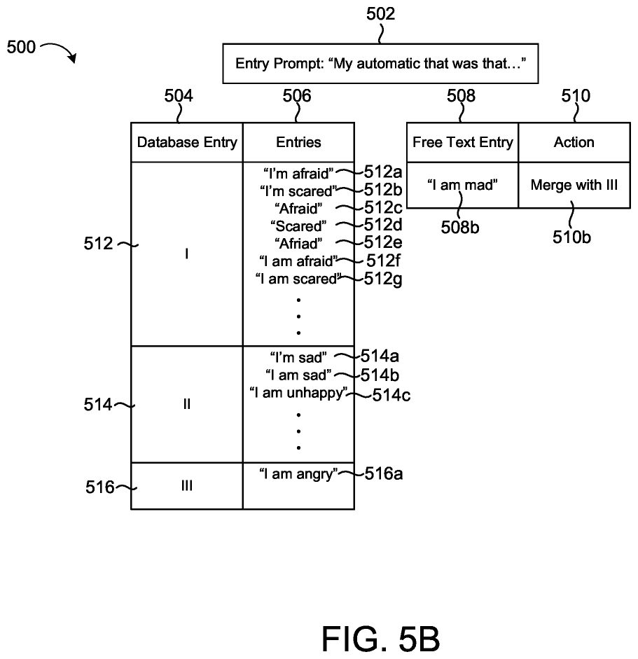

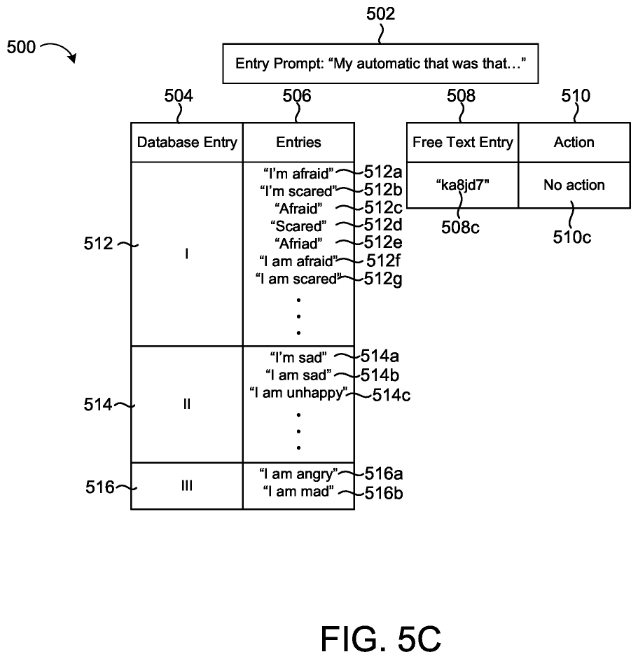

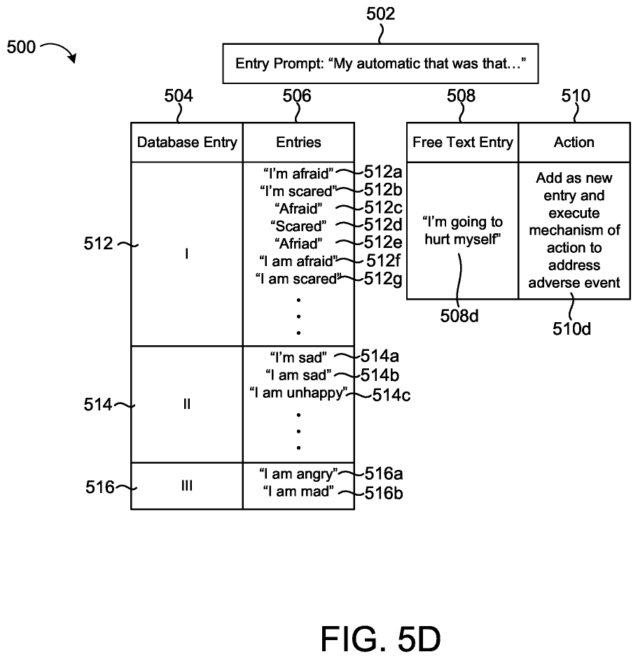

Referring to FIGS. 5A-5D, an exemplary graphical representation of a process 500 for curating the clinically curated database 220 using the system 200 is generally illustrated. The process 500 includes an entry prompt 502, which in some examples may be “My automatic thought was that . . . .” The process 500 includes a plurality of database groups 504, which, in one example, includes a first group 512 and a second group 514. The process 500 includes entries 506 associated with each group 504. For example, the first group 512 includes entries 512a-512g and the second group 514 includes entries 514a-514c. The process 500 is configured to receive (e.g., through the free text entry module 230b) a free text entry 508, e.g., a first free text entry 508a corresponding to “I am angry.” In response to the free text entry 508, the free text review module 250 is configured to determine an action 510. For example, as shown in FIG. 5A, in response to the first free text entry 508a, the free text review module 250 determines a first action 510a corresponding to adding the first free text entry 508a as a new entry to the clinically curated database 220, e.g., through the add entry module 262.

Referring to FIG. 5B, a third database group 516 has been added to the database group 504 with the third group 516 including a first entry 516a corresponding to “I am angry,” that was previously added. The free text entry module 230b is configured to receive a second free text entry 508b corresponding to “I am mad.” The free text review module 250 is configured to determine that the second free text entry 508b is sufficiently similar to the first entry 516a corresponding to “I am angry.” Accordingly, the free text review module 250 determines a second action 510b corresponding to merging the second free text entry 508b with the third group 516, e.g., through the merge entry module 264.

Referring to FIG. 5C, the third group 516 includes a second entry 516b corresponding to “I am mad,” that was previously merged with the third group 516. The free text entry module 230b is configured to receive a third free text entry 508c corresponding to “ka8jd7.” The free text review module 250 is configured to determine that the third free text entry 508c is nonresponsive to the entry prompt 502. Accordingly, the free text review module 250 determines a third action 510c corresponding to taking no action with the third free text entry 508c, e.g., through the no action module 266.

Referring to FIG. 5D, the free text entry module 230b is configured to receive a fourth free text entry 508d corresponding to “I'm going to hurt myself” The free text review module 250 is configured to assign a high risk-assessment value to the fourth free text entry 508d, indicating a high likelihood that an adverse event has occurred or is going to occur. Accordingly, the free text review module 250 determines a fourth action 510d corresponding to adding the fourth free text entry 508d as a new entry to the clinically curated database 220, e.g., through the add entry module 262, and executing a mechanism of action to address the adverse event indicated by the fourth free text entry 508d, e.g., through the regulatory review module 270.

In some implementations, as set forth above, artificial intelligence and/or machine learning may be utilized for various features, functions, components, processes, modules (e.g., the free text review module 250, the clinical data curation module 260, and/or the regulatory review module 270), etc., of the system 200. For example, the free text data 234 may be compared to a dictionary or database (e.g., the externally available data 210 and/or the clinically curated database 220) containing certain keywords (e.g., trademarks, company names, drug names, etc.) to trigger escalation of the free text data 234. Such an escalation may be leveraged by implementing fuzzy matching to compare the free text data 234 to entries in the externally available data 210 and/or the clinically curated database 220. In some implementations, the fuzzy matching processes may include a relatively high sensitivity setting to flag or identify free text data 234 as probable matches with an entry/entries in the externally available data 210 and/or the clinically curated database 220 to escalate such free text data 234. This escalation of the particular free text data 234 may be reviewed by humans (e.g., clinicians, healthcare providers, third-party services, etc.), artificial intelligence, machine learning, etc., to verify a match between the free text data 234 and the entry in the database. Once verified, the fuzzy matches between the free text data 234 and the entry in the externally available data 210 and/or the clinically curated database 220 may be added/merged to the clinically curated database 220 such that upon a subsequent entry of the same free text data 234, that particular free text data 234 may be automatically categorized as a match for escalation.

In some implementations, the clinically curated database 220 may be pre-populated with a base dataset of classifications that are completed and verified by a human (e.g., clinicians, healthcare providers, third-party services, etc.), the classifications including at least “Add Entry,” “Merge Entry,” “No Action,” and “Adverse Event.” This dataset may be used to feed a Natural Language Processing (NPL) text classification model to pre-sort free text data 234 into one of the classifications. In some implementations, the free text data 234 that is pre-sorted into the “Adverse Event” classification may be prioritized for manual review and potential escalation to provide greater emphasis on free text data 234 that may indicate that an adverse event has occurred or is going to occur. For free text data 234 that is not pre-sorted into the “Adverse Event” classification, fuzzy matching or other defined rules may be implemented to determine whether to add the entry, merge the entry, or take no action. In some implementations, these entries may subsequently be reviewed by a human (e.g., clinicians, healthcare providers, third-party services, etc.) for verification. Verified data may then be added/merged with entries in the clinically curated database 220 to train the model and improve accuracy over time.

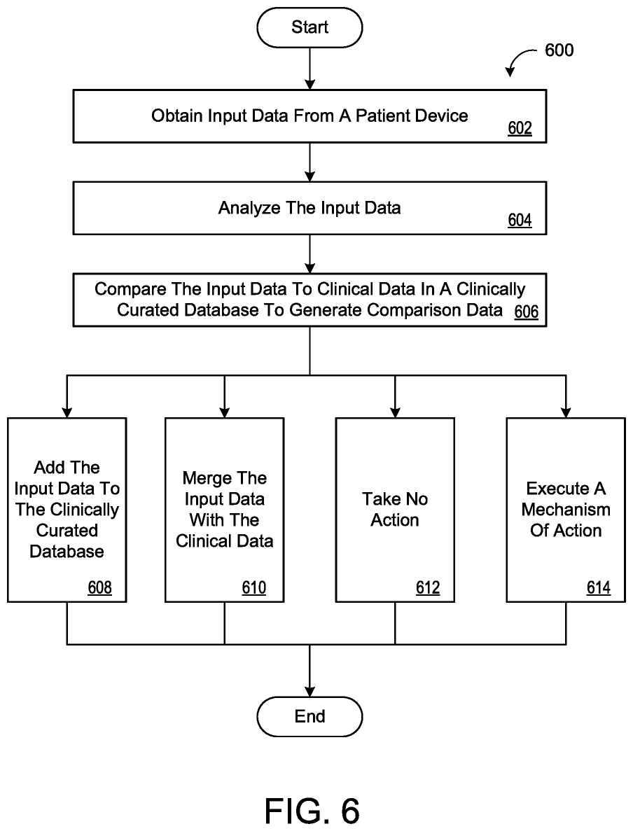

FIG. 6 illustrates a flowchart of a method 600 as set forth herein. At 602, the method 600 includes obtaining input data from a patient device associated with a patient, the input data including free text data generated by the patient. At 604, the method 600 includes analyzing the input data. At 606, the method 600 includes comparing the input data to clinical data in a clinically curated database to generate comparison data. Based on the comparison data, the method 600 executes at least one of steps 608-614. At 608, the method 600 includes adding the input data to the clinically curated database when the comparison data indicates that the input data meets predetermined relevancy criteria and is sufficiently different from the clinical data. At 610, the method 600 includes merging the input data with the clinical data when the comparison data indicates that the input data meets predetermined relevancy criteria and is sufficiently similar to the clinical data. At 612, the method 600 includes taking no action when it is determined that the input data does not meet predetermined relevancy criteria.

In some implementations, the method 600 includes assigning a risk-assessment value to the input data corresponding to the likelihood that the input data indicates an adverse event has occurred or is going to occur. When the risk-assessment value associated with the input data exceeds a predetermined threshold, the method 600 includes executing a mechanism of action at 614. For example, the mechanism of action may include sending an alert to a healthcare provider device associated with a healthcare provider supervising the patient, sending an alert to a call center device associated with a call center, and/or sending an alert to the patient device.

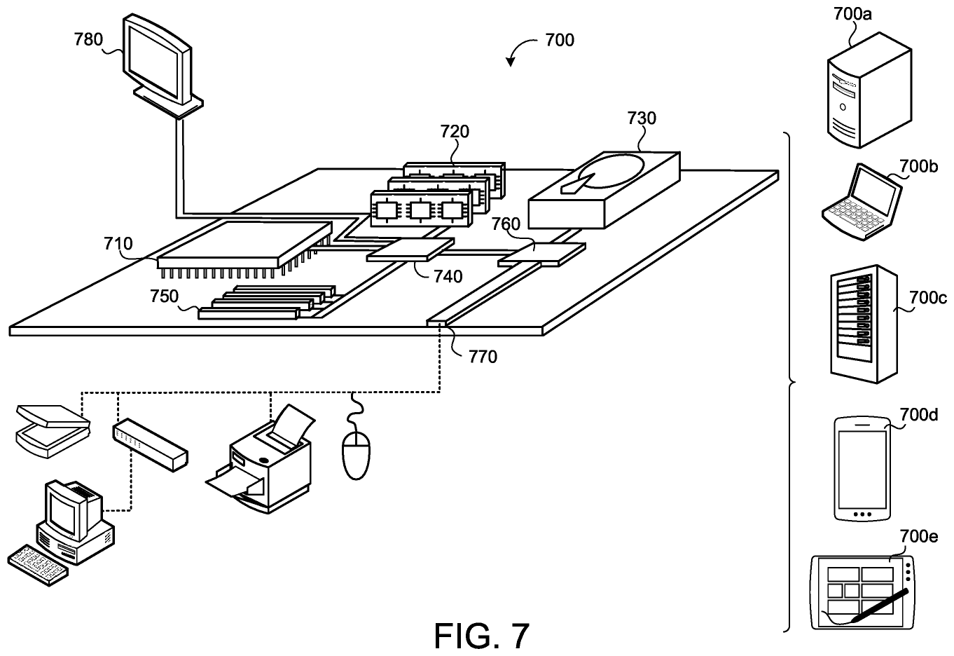

FIG. 7 is schematic view of an example electronic device 700 (e.g., a computing device) that may be used to implement the systems and methods described in this document. The electronic device 700 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The electronic device 700 includes a processor 710, memory 720, a storage device 730, a high-speed interface/controller 740 connecting to the memory 720 and high-speed expansion ports 750, and a low speed interface/controller 760 connecting to a low speed bus 770 and a storage device 730. Each of the components 710, 720, 730, 740, 750, and 760, is interconnected using various busses, and may be mounted on a common motherboard or in other manners as appropriate. The processor 710 can process instructions for execution within the electronic device 700, including instructions stored in the memory 720 or on the storage device 730 to display graphical information for a graphical user interface (GUI) on an external input/output device, such as display 780 coupled to high speed interface 740. In other implementations, multiple processors and/or multiple buses may be used, as appropriate, along with multiple memories and types of memory. Also, multiple electronic device 700 may be connected, with each device providing portions of the necessary operations (e.g., as a server bank, a group of blade servers, or a multi-processor system).

The memory 720 stores information non-transitorily within the electronic device 700. The memory 720 may be a computer-readable medium, a volatile memory unit(s), or non-volatile memory unit(s). The non-transitory memory 720 may be physical devices used to store programs (e.g., sequences of instructions) or data (e.g., program state information) on a temporary or permanent basis for use by the electronic device 700. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 730 is capable of providing mass storage for the electronic device 700. In some implementations, the storage device 730 is a computer-readable medium. In various different implementations, the storage device 730 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 720, the storage device 730, or memory on processor 710.

The high speed controller 740 manages bandwidth-intensive operations for the electronic device 700, while the low speed controller 760 manages lower bandwidth-intensive operations. Such allocation of duties is exemplary only. In some implementations, the high-speed controller 740 is coupled to the memory 720, the display 780 (e.g., through a graphics processor or accelerator), and to the high-speed expansion ports 750, which may accept various expansion cards (not shown).

The electronic device 700 may be implemented in a number of different forms, as shown in FIG. 7. For example, it may be implemented as a standard server 700a or multiple times in a group of such servers 700a, as a laptop computer 700b, as part of a rack server system 700c, as a smartphone 700d, or as a tablet computer 700e.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

As used herein, the term “module” may refer to hardware, software, firmware, or any combination thereof. The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A system comprising:
data processing hardware; and
memory hardware in communication with the data processing hardware, the memory hardware storing instructions that when executed on the data processing hardware cause the data processing hardware to perform operations comprising:
obtaining input data from a patient device associated with a patient, the input data including free text data generated by the patient;
analyzing the input data to determine whether the input data meets predetermined relevancy criteria; and
comparing the input data to clinical data in a clinically curated database to generate comparison data, and based on the comparison data, performing at least one of the following curation operations:
adding the input data to the clinically curated database when the input data meets predetermined relevancy criteria and when the comparison data indicates that the input data is sufficiently different from the clinical data;
merging the input data with the clinical data when the input data meets predetermined relevancy criteria and when the comparison data indicates that the input data is sufficiently similar to the clinical data; and
taking no action when it is determined that the input data does not meet predetermined relevancy criteria.

2. The system of claim 1, wherein the operations further comprise analyzing the input data to determine a likelihood of an adverse event, and assigning a risk-assessment value to the input data corresponding to the likelihood that the input data indicates an adverse event has occurred or is going to occur.

3. The system of claim 2, wherein when the risk-assessment value associated with the input data exceeds a predetermined threshold, the operations further comprise executing a mechanism of action to address the adverse event.

4. The system of claim 3, wherein the mechanism of action includes sending an alert to a healthcare provider device associated with a healthcare provider supervising the patient, the alert indicating that an adverse event has occurred or is going to occur.

5. The system of claim 3, wherein the mechanism of action includes sending an alert to a call center device associated with a call center, the alert indicating that an adverse event has occurred or is going to occur and the alert providing instructions to the call center to contact the patient via the patient device.

6. The system of claim 3, wherein the mechanism of action includes sending an alert to the patient device, the alert providing information to the patient to address the adverse event.

7. The system of claim 1, wherein analyzing the input data and comparing the input data to the clinical data are performed by implementing artificial intelligence.

8. The system of claim 7, wherein the artificial intelligence is supervised by a healthcare professional.

9. The system of claim 7, wherein the artificial intelligence includes unsupervised machine learning.

10. The system of claim 1, wherein the input data is input in response to an inquiry, and the predetermined relevancy criteria is satisfied when the input data is responsive to the inquiry.

11. A method comprising:
obtaining, via one or more processors, input data from a patient device associated with a patient, the input data including free text data generated by the patient;
analyzing, via the one or more processors, the input data to determine whether the input data meets predetermined relevancy criteria; and
comparing, via the one or more processors, the input data to clinical data in a clinically curated database to generate comparison data, and based on the comparison data, performing, via the one or more processors, at least one of the following curation operations:
adding the input data to the clinically curated database when the input data meets predetermined relevancy criteria and when the comparison data indicates that the input data is sufficiently different from the clinical data;
merging the input data with the clinical data when the input data meets predetermined relevancy criteria and when the comparison data indicates that the input data is sufficiently similar to the clinical data; and
taking no action when it is determined that the input data does not meet predetermined relevancy criteria.

12. The method of claim 11, further comprising analyzing the input data to determine a likelihood of an adverse event, and assigning a risk-assessment value to the input data corresponding to the likelihood that the input data indicates an adverse event has occurred or is going to occur.

13. The method of claim 12, further comprising executing a mechanism of action to address the adverse event when the risk-assessment value associated with the input data exceeds a predetermined threshold.

14. The method of claim 13, wherein the mechanism of action includes sending an alert to a healthcare provider device associated with a healthcare provider supervising the patient, the alert indicating that an adverse event has occurred or is going to occur.

15. The method of claim 13, wherein the mechanism of action includes sending an alert to a call center device associated with a call center, the alert indicating that an adverse event has occurred or is going to occur and the alert providing instructions to the call center to contact the patient via the patient device.

16. The method of claim 13, wherein the mechanism of action includes sending an alert to the patient device, the alert providing information to the patient to address the adverse event.

17. The method of claim 11, wherein analyzing the input data and comparing the input data to the clinical data are performed by implementing artificial intelligence.

18. The method of claim 17, wherein the artificial intelligence is supervised by a healthcare professional.

19. The method of claim 17, wherein the artificial intelligence includes unsupervised machine learning.

20. The method of claim 11, wherein the input data is input in response to an inquiry, and the predetermined relevancy criteria is satisfied when the input data is responsive to the inquiry.
